(19) United States
(12) Patent Application Publication
(10) Pub. No.: US 2022/0288583 A1
(43) Pub. Date: Sep. 15, 2022

(54) A SYSTEM AND METHOD FOR COLLECTING, SAMPLING, CULTURING AND INSPECTING BIOLOGICAL FLUIDS

(71) Applicant: NOVAMED LTD., Jerusalem (IL)

(72) Inventors: Emil KATZ, Savyon (IL); Gadi PORATH, Kiryat Tivon (IL)

(21) Appl. No.: 17/629,940

(22) PCT Filed: Jul. 29, 2020
(86) PCT No.: PCT/IL2020/050837

§ 371 Date: Jan. 25, 2022

(30) Foreign Application Priority Data
Jul. 31, 2019 (IL) 268395

(51) Int. Cl. B01L 3/00; A61B 10/00

(52) U.S. Cl. CPC B01L 3/50825; A61B 10/007; B01L 2200/025; B01L 2300/042; B01L 2300/046

(57) ABSTRACT

A system for collection, sampling, culturing, and inspection of fluids. The system includes a culture testing surface, suitable to be read in a reading apparatus. The testing surface includes stabilization element(s), suitable to maintain the surface in a desired position in a plate adapted to be processed by the reading apparatus.

### FIELD OF THE INVENTION

The present invention is in the field of biological inspection equipment. More particularly, the invention relates to a system and method for collection, sampling, culturing and inspection of biological fluids.

### BACKGROUND OF THE INVENTION

Throughout the years, the accumulated medical knowledge, and advanced diagnosis protocols in particular, have increased the demand for laboratory inspections of biological samples. The continuous technological developments in the field of biological testing methods, along with the developments of computerized equipment and computer and networking capabilities for handling mass data provide laboratories with improved tools for handling large quantities of biological inspections.

Nowadays, when a patient needs to use a laboratory services for testing a biological fluid sample, for example, for revealing infections, such UTI (Urinary Tract Infection), including revealing the specific pathogen(s) and performing colonies counting, it usually takes a few days until the results are back. The process usually includes delivering the sample to a laboratory where biological fluids are collected, sampled, prepared for testing, and tested. Sometimes samples are delivered to a clinic, often in a closed cup or in an evacuated test tube, and then transferred to a collection center and continue to a laboratory where they are being tests. Additionally, the inspection of fluid samples for the presence and enumeration of microorganisms requires additional steps, such as using a manual inoculating loop or an automatic inoculating device, streaking in isolation the withdrawn fluid sample into a culture testing surface, such as a in Petri plate, and incubation. The culturing process, including screening the surface, can take several days.

While waiting for test results, a physician may prescribe preventive medicines, such as antibiotic. In other cases, a physician may avoid treatment until he gets the results back from the lab, which can cause a delay in necessary treatment or escalation in the condition of a sick patient. Faster laboratory results could provide an earlier distinction regarding the presence of contaminations and the condition of patients, thereby assist physicians to provide appropriate treatment earlier and avoid unnecessary treatments.

When referring to lab tests that include a culturing process, one way to shorten the period of time between providing a sample and receiving the test results, is to begin the culturing process as soon as possible, when giving the sample. Further shortening potential is in automating the testing operation, performing rapid screening and answering.

It is an object of the present invention to provide a system and method that allow reducing the testing stages of biological fluid samples and begin the inoculation and the incubation earlier at the POC (Point of Care) before the sample even reaches the Lab.

It is another object of the present invention to provide a system and method for collecting, sampling, culturing, and inspecting biological fluids, suitable to be operated without the need to touch the testing components.

Other objects and advantages of the invention will become apparent as the description proceeds.

It should be noted that although the description refers to biological fluids or urine samples that are tested mostly for contaminations, the system and method of the invention can be applied to the use of any fluids that need to be tested, and many testing processes can be integrated with the system and method of the invention, as can be easily concluded from the description by an average person skilled in the art.

### SUMMARY OF THE INVENTION

The invention relates to a system for collection, sampling, culturing, and inspection of fluids, comprising a culture testing surface, suitable to be read in a reading apparatus, wherein the surface comprises at least one stabilization element, suitable to maintain the surface in a desired position in a plate adapted to be processed by the reading apparatus.

The reading apparatus can be of any type suitable to give an output of test results. For example, the apparatus can be a digital reading apparatus, such as a digital reading apparatus that is common in the field of reading lab tests. Another example of a reading apparatus is a combination of an image acquisition apparatus and image processing means. Of course, the reading apparatus can be of any other type suitable to provide interpretation of test results, based on the testing surface.

The system according to the invention comprises a fluid collection container, provided with a lid, into which a user provides fluids that need to be tested. The system also comprises a sampling and culturing apparatus, comprising a vessel, a cap, and an inoculating element. The culture testing surface is suitable to be inserted through an opening in the lid of the fluid collection container, and it is releasably attached to the cap of the sampling and culturing apparatus. The culture testing surface is adapted to be inserted into the vessel of the sampling and culturing apparatus, and to be detached from the cap of the sampling and culturing apparatus.

The inoculating element comprises prongs. When performing a test, the prongs are dipped into the fluids that need to be tested, and then come in contact with the testing surface, scratching the surface as it is being inserted into the vessel of the culturing apparatus, thus performing a streaking and inoculating activity.

The lid of the fluid collection container comprises an opening for an evacuation test tube. It can be used, for example, when there is a need to extract fluids from the fluid collection container in sterile conditions, without opening the lid of the container. The system, according to another embodiment of the invention, further comprise a cover for the tube opening. According to another embodiment of the invention, the system also comprise a cover for the second opening located in the lid of the fluid collection container.

According to one embodiment of the invention, the cap of the sampling and culturing apparatus and the culture testing surface comprise connection elements, suitable to connect the cap to the culture testing surface. According to this embodiment, the cap of the sampling and culturing apparatus also comprises at least one releasing element, suitable to release the culture testing surface from the cap. The releasing elements can be used, for instant, when placing the testing surface on a scanning surface. The cap of the culturing apparatus further comprises gripping elements, suitable to grip the inoculating element.

According to one embodiment of the invention, the stabilization elements are wing-like strips that are attached to the culture testing surface. According to another embodiment of the invention, the stabilization elements are a part of the culture testing surface. The shape and size of the testing surface can also be referred to as a form of stabilization elements, for example, when it is essentially the same size or length of the scanning surface, thus preventing the movement of the testing surface in relation to the scanning surface. An example of a common scanning surface a petri plate.

The culture testing surface, according to one embodiment of the invention, is divided into two or more sections, thus providing the ability to perform more than one test at a time. Each part of the divided surface can be used for different tests that are sampled, streak, and scanned simultaneously.

According to another embodiment of the invention, the lid of the fluid collection container further comprises an opening adapted to receive a strip inserted therethrough. The strip can be used for preliminary and/or additional tests. The strip opening can also be provided with a one-way valve, suitable to be inserted with the strip through it and into the fluid collection container without removing the lid.

According to another embodiment of the invention, the vessel of the sampling and culturing apparatus comprises a projection at its top internal section, geometrically suitable to allow taking out the culture testing surface together with the inoculating element from the vessel, and forcing inoculating element to stay at the top section of the vessel when inserting the culture testing surface back into the vessel.

The invention also relates to a method for collection, sampling, culturing, and inspection of fluids, comprising:
a) providing a culture testing surface, suitable to be read in a reading apparatus, wherein the surface comprises at least one stabilization element, suitable to maintain said surface in a desired position in a plate or on a scanning surface, adapted to be processed by the reading apparatus;
b) providing a fluid collection container provided with a lid, wherein the culture testing surface is suitable to be inserted through an opening in the lid of said fluid collection container;
c) providing a sampling and culturing apparatus, comprising a vessel, a cap, and an inoculating element, wherein the culture testing surface is releasably attached to the cap of the sampling and culturing apparatus, and wherein the culture testing surface is adapted to be inserted into the vessel of the sampling and culturing apparatus, and to be detached from the cap of the sampling and culturing apparatus;
d) positioning said inoculating element at the bottom section of said culture testing surface;
e) holding the cap of the sampling and culturing apparatus while it is attached to the culture testing surface along with the inoculating element, and pulling them out of the vessel of the sampling and culturing apparatus;
f) dipping the prongs of the inoculating element in a fluid sample; and
g) returning the assembly of the inoculating element, the culture testing surface, and the cap of the sampling and culturing apparatus back into the vessel of the sampling and culturing apparatus.

According to another embodiment of the invention, the method further comprises performing an incubation process on the sampling and culturing apparatus containing the culture testing surface.

According to another embodiment of the invention, the method further comprises detaching the culture testing surface from the cap of the sampling and culturing apparatus and from the inoculating element, and placing it on a surface, such as a petri plate.

According to another embodiment of the invention, the method further comprises placing the culture testing surface in a reading apparatus, thereby allowing the reading apparatus to read a value relative to the status of the culture testing surface.

The invention also relates to an apparatus including:
(a) a cap with a reversible pivotal attachment socket and two engagement tabs on an inner surface thereof;
(b) an inoculating surface containing culture media and having a reversible pivotal attachment axle at a proximal end and two normally open flexible wings; and
(c) an inoculating element adapted to slideably travel from the distal end to the proximal end causing said wings to close and engage said engagement tabs. In some embodiments the apparatus includes a flexible rhomboid member at one or both ends of the inoculation surface.

The Invention also relates to a method including placing a distal end of an inoculation surface connected to a cap against an inner wall of a petri dish and rotating the cap attached to the inoculation surface about a pivotal attachment axle to disengage a pivotal attachment socket from the axle so that flexible members attached to the inoculating surface secure the inoculating element in the petri dish. In some embodiments the flexible members are configured as wings which assume an open configuration upon disengagement of said axle from said pivotal attachment socket. Alternatively or additionally, in some embodiments the flexible members are configured as rhomboid elements at one or both ends of said inoculation surface.

### BRIEF DESCRIPTION OF THE DRAWINGS

FIG. 1 shows a collection container, according to one embodiment of the invention, suitable to host fluids provided by a user;

FIG. 2 shows a sampling and culturing apparatus, according to one embodiment of the present invention;

FIG. 3 is a front view of the culturing apparatus of FIG. 2, wherein the testing surface is connected to the cap, and the inoculating element is located at the bottom section of the testing surface;

FIG. 4 is a front view of the culturing apparatus of FIG. 2, wherein the testing surface is connected to the cap, and the inoculating element is located at the top section of the testing surface;

FIG. 5A is a perspective of a cap of the culturing apparatus, according to another embodiment of the invention, wherein the cap comprises a grasping tube and gripping elements;

FIG. 5B is a perspective view of a testing surface, according to another embodiment of the invention, wherein the surface comprises hooks;

FIG. 6 is a perspective view of a cap, wherein an inoculating element is attached to said cap by gripping elements;

FIG. 7 shows a testing surface, according to another embodiment of the invention, inside a Petri dish;

FIG. 8A shows a fluids container, which is similar to the container of FIG. 1, but also comprises a strip opening 802;

FIG. 8B shows the container of FIG. 8A, and also hidden lines that indicate the position of a strip inside the container;

FIG. 9 is a front perspective view of an inoculation surface connected to a cap prior to inoculation, with an inset showing details of the connection, according to another exemplary embodiment of the invention;

FIG. 10 is a front perspective view of an inoculation surface connected to a cap prior after inoculation, with an inset showing details of the connection, according to the embodiment of FIG. 9;

FIG. 11 is an exploded front perspective view of the connection between cap and inoculation surface of the embodiment of FIG. 9 and FIG. 10 with an inset showing details of the connection;

FIG. 12 is a top view of the inoculation surface connected to a cap prior after inoculation of FIG. 10 during insertion into a petri dish; and

FIG. 13 is a top view of the inoculation surface of FIG. 12 with cap removed following insertion into a petri dish.

### DETAILED DESCRIPTION OF THE DRAWINGS

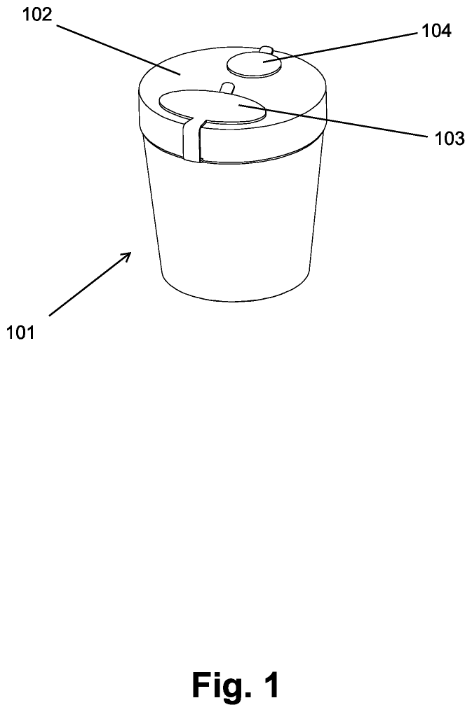

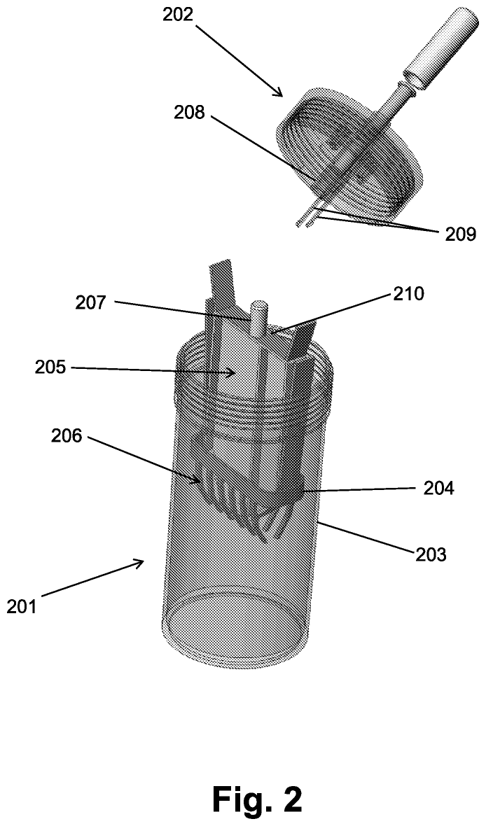

The present invention relates to a system and method for collecting, sampling, culturing, and inspecting fluids, which comprises a fluid collection container and a culturing apparatus. FIG. 1 shows a fluid collection container 101, according to one embodiment of the invention, suitable to host fluids provided by a user, such as a urine sample. Lid 102 of container 101 can be removed in order to open container 101, thus allowing filling container 101 with fluids. After container 101 is filled and closed by a user, it is usually taken to a lab employee, which uses the fluid for testing. One opening of container 101 (not shown) is covered by cover 103, which is suitable to be removed, thus revealing the opening, and a testing surface (such as surface 205 that is shown in FIG. 2) can be inserted into container 101 through the opening that is located under cover 103, in order to perform tests. Container 101 also comprises an opening for evacuating test tube, which is located under second cover 104, according to one embodiment of the invention. Both covers 103 and 104 can be a removable sticky label, according to one embodiment of the invention, and cover 104 leads to a round port with a short needle (not shown) for withdrawing fluids into an evacuated test tube, for chemical lab testing. Chemical results are usually analyzed within several hours after receiving them in the lab.

FIG. 2 shows a sampling and culturing apparatus 201, according to one embodiment of the present invention, which comprises cap 202, vessel 203, and streaking and inoculating element 204 (which can also be referred to as simply “inoculating element”). FIG. 2 also shows testing surface 205 that is divided into two culture surfaces with different agar mediums. It should be noted that the testing surface, according to the present invention, such as surface 205, can be divided into any number of surfaces, which can be suitable to test different parameters, mix flora and/or cross results for better analysis, or not divided at all. In order to perform the testing of fluids, prongs 206 of inoculating element 204 need to come in contact with said fluids, and then streak surface 205, as will be described along the following description.

Surface 205 is initially attached to cap 202, and in its initial state inner tube 207 of surface 205 is located inside outer tube 208 of cap 202, and pins 209 are located inside hole 210 of surface 205, thus connecting surface 205 to cap 202. Inoculating element 204 is initially located at the bottom of surface 205. When performing a test, which includes dipping, streaking, and isolating the sample, a lab operator takes out the assembly of surface 205 and cap 202, along with the attached components, including element 204, from vessel 203 and dips only prongs 206 of element 204 in the fluid that is inside container 101, without dipping surface 205 as well. Surface 205 is inserted into container 101 through the opening of container 101 that is located under cover 103 for sampling fluids. FIG. 2 is actually an exploded view of the components of culturing apparatus 201 as detailed above.

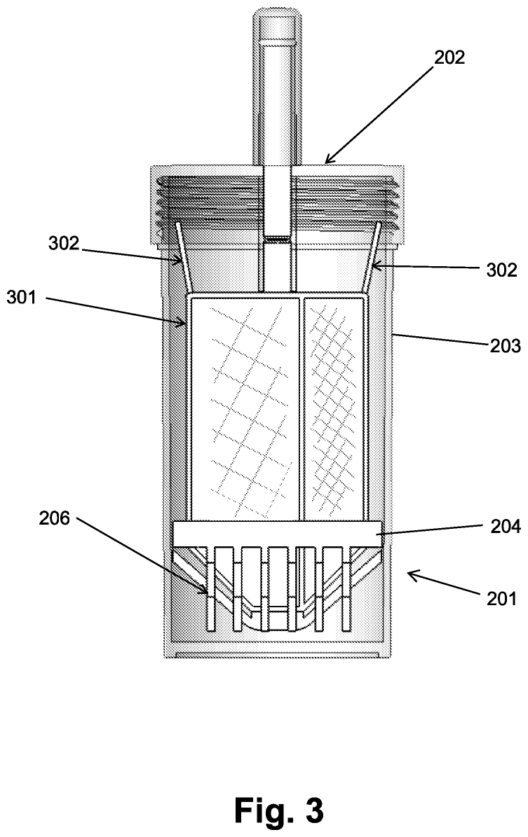

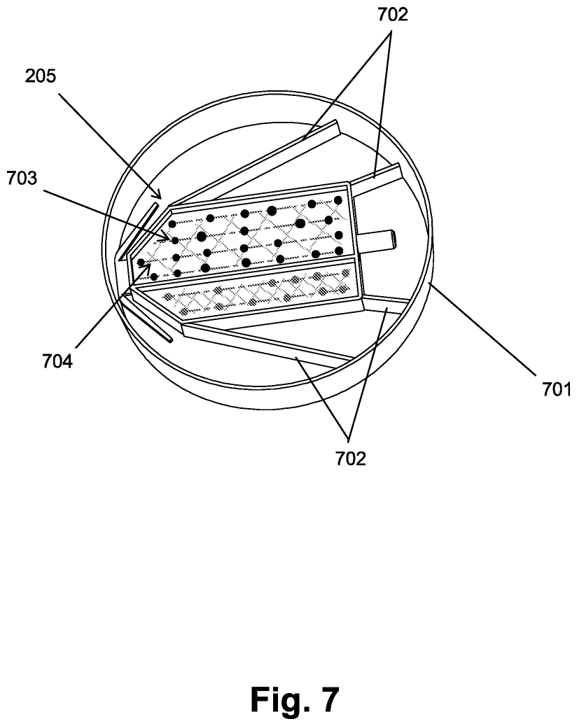

FIG. 3 is a front view of culturing apparatus 201 of FIG. 2, comprising testing surface 301, according to another embodiment of the invention, wherein surface 301 is connected to cap 202. When a lab employee receives a sample container from a patient, potentially at the POC or from home, such as container 101 of FIG. 1, he first needs to open cover 103 (that is shown in FIG. 1) of container 101, and then disconnect cap 202 from vessel 203 (possible, for example, by a screw motion), take out the assembly of cap 202 and surface 301, and dip only prongs 206 of element 204 in the fluid sample. After that he takes the assembly of cap 202 and surface 301, and inserts it back into the vessel 203. FIG. 3 also shows wings 302, which are held in place by cap 202. The function of the wings, such as wings 302, will be explained in detail along the description when referring to FIG. 7.

Vessel 203 comprises a small projection at its top internal section (not shown), geometrically suitable to allow a user to take out the assembly of cap 202 and surface 301 together with element 204 from vessel 203, and forcing element 204 to stay at the top section of vessel 203 when inserting surface 301 back into vessel 203.

FIG. 3 also shows that surface 301 comprises diagonal lines. Those lines are only for the purpose of illustrating that there is a difference between the two sections of surface 301, for example, the fact that each surface can be suitable for different types of test and agar mediums. Of course that it is only optional and surface 301 can be divided into any number of different or identical sections (or a combination of a number of different sections and a number of identical sections).

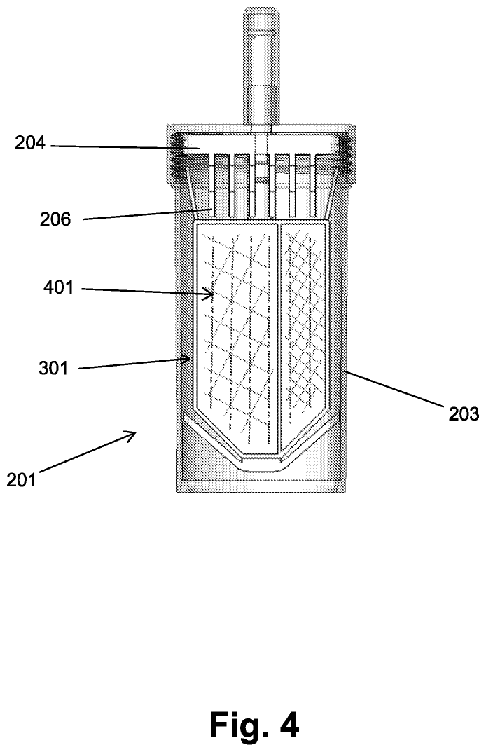

FIG. 4 is also a front view of apparatus 201, wherein surface 301 is shown after the streaking process, so that inoculating element 204 is located at the top section of vessel 203, showing the state of the components of apparatus 201 after inserting said components back into vessel 203. Since inoculating element stays at the top section of vessel 203 while surface 301 is being inserted into vessel 203, the wet prongs 206 come in contact with surface 301 and scratch surface 301 as it is being inserted into vessel 203, thus performing a streaking and inoculating activity. It is customary to do so in isolation. Prongs 206 that came in contact with the fluids that are tested streak testing surface 301, inserting the fluids into surface 301. Streaking lines, such as line 401, are shown in FIG. 4. After the streaking process, culturing apparatus 201 is taken into incubation.

According to another embodiment of the invention, the system further comprises patient identification elements, suitable to tag the sample of a patient in the container and also in the culturing apparatus. The identification element can be, for example, a barcode label.

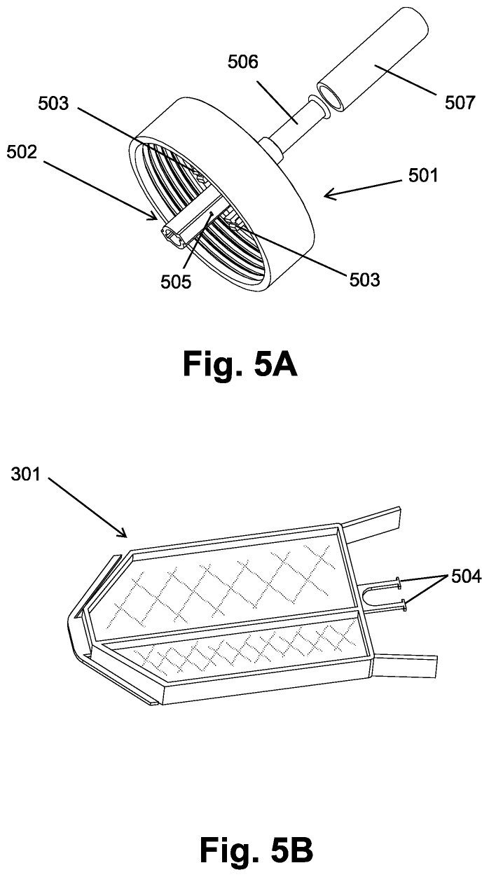

FIG. 5A is a perspective of cap 501 of the culturing apparatus, according to another embodiment of the invention, wherein cap 501 comprises grasping tube 502 and gripping elements 503. FIG. 5B is a perspective view of testing surface 301, wherein surface 301 comprises flexible hooks 504, suitable to be inserted into grasping tube 502 of cap 501 and to be fixed within designated holes, such as hole 505. Although FIG. 5A shows only one hole 505, tube 502 comprises two holes (the other one on the other side of tube 502 that is not visible in FIG. 5A), suitable to fixate both hooks 504 inside tube 502. Gripping elements 503 are suitable to grip the inoculating element (such as element 204 that is shown in previous figures) when it is located at the top section of the vessel of the culturing apparatus (such as vessel 203 that is shown in previous figures), when inserting the components back into the vessel. The peripheral section of the inoculating element is suitable to be inserted into gripping elements 503 and held by them.

After the inoculating element is fixated to cap 501 by gripping elements 503, cap 501 can be detached from surface 301, and surface 301 can be placed in a petri plate or on any other different surface suitable to go into scanning. The detachment of cap 501 from surface 301 can be performed by any mechanical means suitable to force hooks 504 out of designated holes, such as hole 505 of tube 502, for example, by an external push pin, such as pin 506. Pin 506 is located in cap 501 and can press flexible hooks 504 out of holes 505 and release surface 301 from cap 501. Cap 501 also comprises pin cover 507, suitable to prevent pressing pin 506 by mistake while handling the system and to provide a closed system before using it.

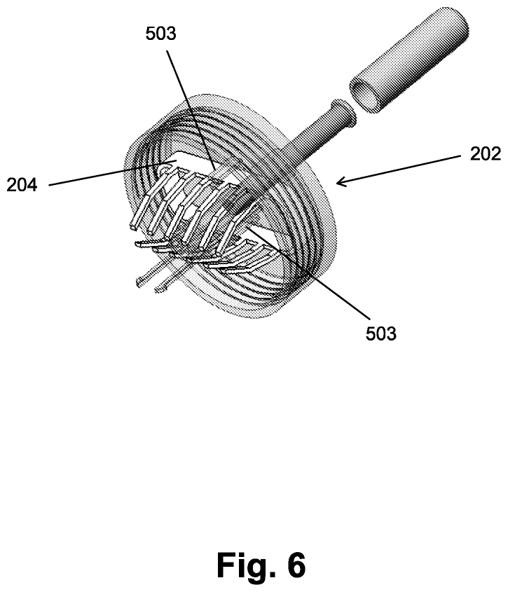

FIG. 6 is a perspective view of cap 202, as the one of FIGS. 2, 3 and 4, after the streaking process and the detachment of inoculating element 204 from the testing surface, wherein inoculating element 204 is attached to cap 202 by gripping elements 503.

FIG. 7 is a perspective view of testing surface 205 of FIG. 2, after it has been placed inside a petri plate 701. The placement of surface 205 on a plate allows performing screening of surface 205, for example a digital screening by a digital reader, or any other suitable means for different types of scans. Another example for reading the results is by taking a picture of the surface by an image acquisition apparatus, and then performing image processing in order to get the results. According to this specific embodiment of the invention, testing surface 205 is geometrically suitable to be places inside a common petri plate, but of course that the plate can be replaced with any other screening surface, and the size and shape of surface 205 can be adjusted to said screening surface accordingly. Identification elements can also be provided on the screening surface, such as a barcode label, consisting patient identification details or any other desired information. In addition, after placing surface 205 into petri plate 701, surface 205 can undergo an incubation process, prior to the screening process, while located on the plate.

As shown in FIG. 7, surface 205 comprises wings 702. After placing surface 205 in plate 701, wings 702 are used as stabilization elements, suitable to prevent surface 205 from moving inside plate 701. The flexibility of wings 702 and their tendency to move toward the edges of plate 701 can be obtained by different mechanical method, for example, by some injection molding technics. The friction between wings 702 and plate 701 prevents the movement of surface 205 in relation to plate 701.

When surface 205 is attached to a cap of a culturing apparatus, wings 702 are held inside said cap, as shown in the embodiment of FIG. 3, wherein wings 302 are restricted by cap 202. Wings 702 are flexible and suitable to hold surface 205 in place when surface 205 is placed on a screening surface, such as plate 701. The testing surface of the culturing apparatus according to the invention can comprise any number of wings to hold it in place while positioned on a screening surface or inside any other element.

According to another embodiment of the invention, the length of the testing surface can be essentially the same length as the screening surface, thus preventing its movement in relation to the surface. Of course, other stabilization elements suitable to prevent the movement of the testing surface in relation to the screening surface, can be used while performing, for example, digital marking of the colonies and automatic specific colony pick-up for additional tests, such as sensitivity, as will be obvious to a person skilled in the art.

When the testing surface is divided, more than one test can be performed on the same plane at the same time. The screening process includes rapid identification of microorganisms and identification of mix pathogens, usually by color, and the scanning can be performed on multiple surfaces. This fact is extremely important when the testing of the surface is performed by scanning, since it would be possible to scan only once and get several results, from different surfaces, at the same time.

The system and method of the invention provide a process that does not include manual handling of the components that come in contact with the sample. The lab operator touches only the cap of the culturing apparatus, when dipping the prongs of the inoculating element inside a sample, when inserting the components back into the vessel of the culturing apparatus, and also when placing the testing surface onto or inside a petri plate, or the like. Thus, minimizing contaminations and other pathogens that can influence clinical results, and reducing the waiting time for tests results.

FIG. 7 also shows a typical colony on surface 205, illustrated by dots, such as dot 703, on surface 205. Those dots are located along lines 704, which are created as a result of the streaking process, as shown in FIG. 4, wherein such lines are marked by numeral 401. FIG. 7 is an illustration of surface 205 after an incubation process, which resulted in colony growth, at a position suitable for the screening stage.

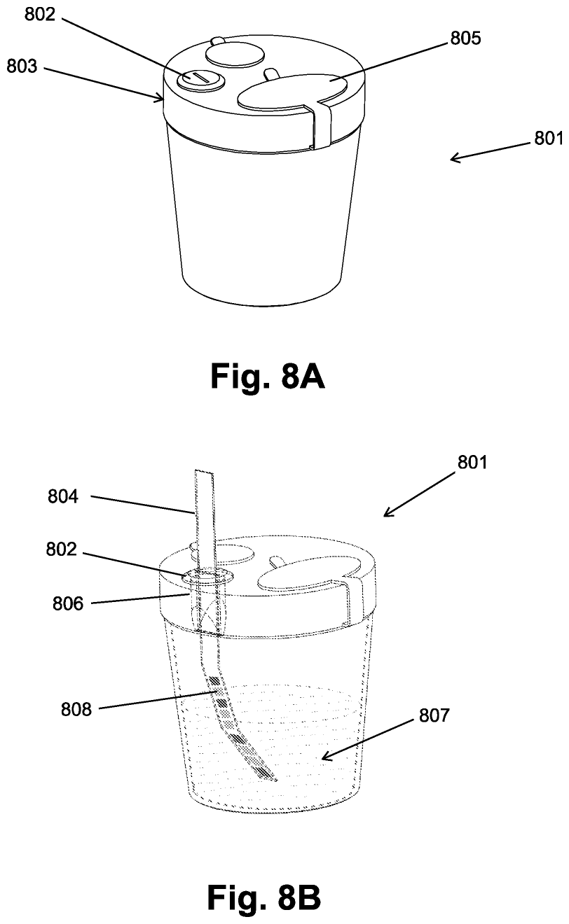

According to another embodiment of the invention, the system also comprises a diagnostic reagent strip for testing fluids, for easy inserting and dipping in the fluids that are inside the collection container. FIG. 8A shows container 801, which is similar to container 101 of FIG. 1, but also comprises strip opening 802. Lid 803 of container 801 allows dipping a strip, such as strip 804 of FIG. 8B in the fluids that are inside container 801, without opening lid 803 or surface opening 805. After dipping the strip (usually for 2-3 seconds) it can be pulled out for inspection and/or drying process (which is common to do so by air). When using a color changing strip, results become apparent usually after 60-90 seconds. The visual reading of the colors can be performed, for example, by using a color chart or by using a common digital reader.

Referring to FIGS. 8A and 8B, Strip 804 can be used for additional and rapid testing of fluids, apart from the culture testing that is performed by using a testing surface, for example, nitrite levels. Strip 804 can also be used as an indication for using a culture testing surface, for example, strip 804 can be used to test nitrite and/or leukocytes in urine samples, and if the answer is positive, then an operator can also test the urine with the culture testing surface. Strip opening 802 of lid 803 can comprise a one-way duck-bill valve, such as valve 806 of FIG. 8B, which is typically made of silicone or rubber, according to one embodiment of the invention. Of course, valve 806 can be replaced with other type of valves that prevent fluids from dripping out from lid 803. FIG. 8B shows container 801 of FIG. 8A and also hidden lines that indicate the position of strip 804 inside container 801, and additional lines that illustrate sample 807. FIG. 8B also shows that strip 804 comprises indicational squares, such as square 808, suitable to change color and thus indicate tests results.

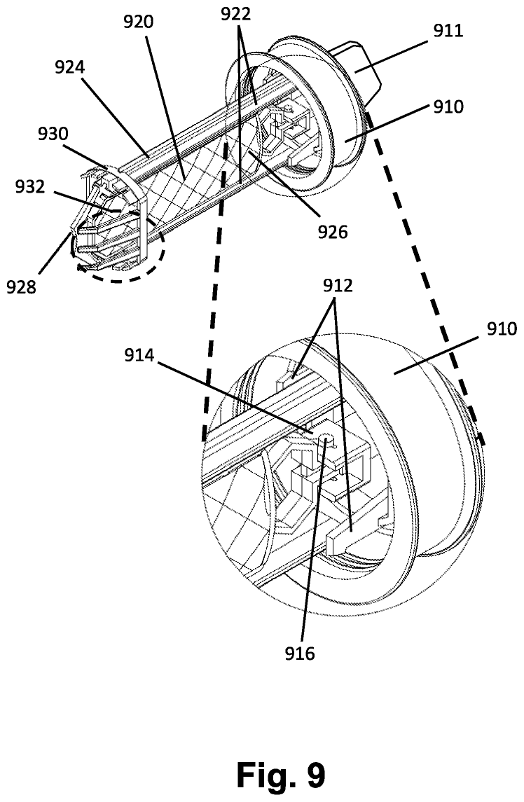

FIG. 9 illustrates another exemplary inoculation tool according to some embodiments of the invention in a pre-inoculation state. In the depicted embodiment, a cap 910 holds a reversible pivotal attachment socket 914 and two engagement tabs 912 on an inner surface thereof. In the depicted embodiment, an inoculating surface 920 containing culture media includes a reversible pivotal attachment axle 916 at its proximal end 926 and two normally open flexible wings 922. In FIG. 9 wings 922 are held closed by cap 910. In the depicted embodiment, inoculating surface 920 has an inoculating element 930 equipped with prongs 932 mounted on it so that tips of prongs 932 are near a distal end 928 of inoculation surface 920. Inoculation element 930 is adapted to slideably travel from distal end 928 to proximal end 926 of inoculating surface 920 allowing inoculation element 930 to engage engagement tabs 912 in cap 910. In some embodiments wings 922 are provided with guide rails 924. Optionally, guide rails 924 contribute to alignment of inoculating element 930 with tabs 912 in cap 910. The depicted assembly of FIG. 9 is functionally similar to the assembly of parts 204/205/206 depicted in FIG. 3 and FIG. 4 in many respects. As with the embodiment depicted in those Figures, the vessel (e.g. 203 in FIG. 3 and FIG. 4) in which the assembly is inserted comprises a small projection at its top internal section geometrically suitable to allow a user to take out the assembly of cap 910 and surface 920 together with element 930 from the vessel, and forcing element 930 to travel from distal end 928 to proximal end 926 when inserting surface 920 back into the vessel. In the depicted embodiment, a handle 911 on cap 910 contributes to ease of manipulation of the cap.

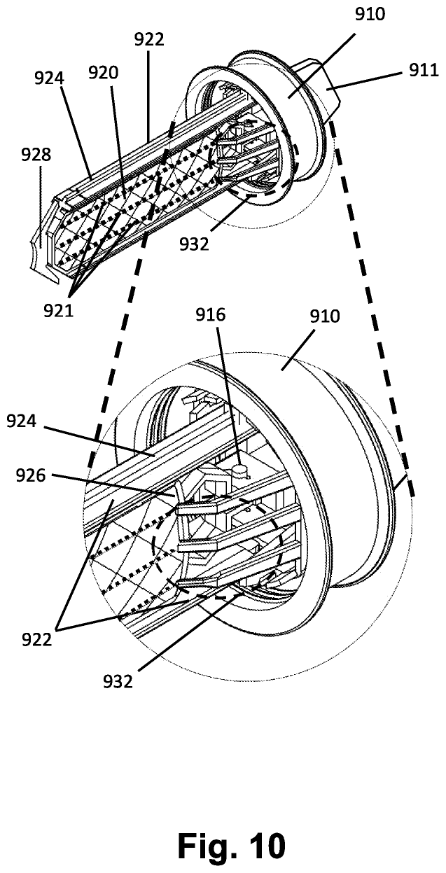

FIG. 10 illustrates the exemplary inoculation tool of FIG. 9 in a post-inoculation state. Numbering of parts in FIG. 10 is as in FIG. 9. In FIG. 10, inoculation element 930 has travelled from distal end 928 to proximal end 926 of inoculation surface 920 and engaged tabs 912 in cap 910. As 930 slides along guide rails 924 of wings 922 it drags tips of prongs 932 along culture media on 920. Tips of prongs 932 form inoculation streaks 921 (dotted lines on 920).

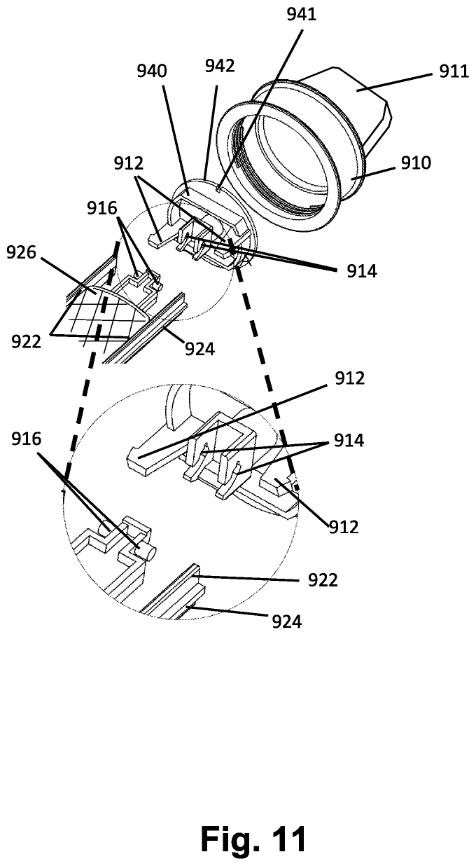

FIG. 11 is an exploded view of the assembly of FIG. 9 and FIG. 10 showing the relationship between attachment socket 914 and attachment axle 916 in greater detail. In the depicted embodiment, attachment socket 914 is mounted on a disk 940 which fits into cap 910. In some embodiments notch 941 on cap 910 dictates orientation of disk 940 with respect to cap 910 by mating with a corresponding ridge (not visible) in cap 910 and prevents rotation of disk 940 with respect to cap 910. The outer form of frame 942 on disk 940 matches the inner form of inoculation element 930 so that 930 engages 942 when it is moved into cap 910 (see FIG. 10). Attachment sockets 914 are sized and positioned to engage and retain axle 916. Cap 910 with disk 940 inserted is assembled with inoculation surface 920 by pushing axle 916 into sockets 914. Engagement of axle 916 with sockets 914 connects inoculation surface 920 to cap 910.

Disconnection of inoculation surface 920 from cap 910 is accomplished by rotating cap 910 by 90° about axle 916 which is connected to inoculation surface 920. This causes release of axle 916 from sockets 914.

In other respects, the depicted assembly of FIG. 9 is functionally different from the assembly of parts 204/205/206 depicted in FIG. 3 and FIG. 4.

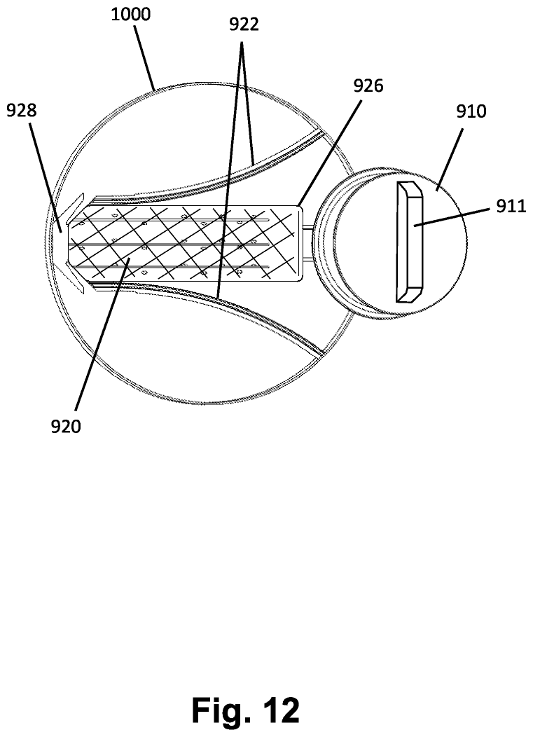

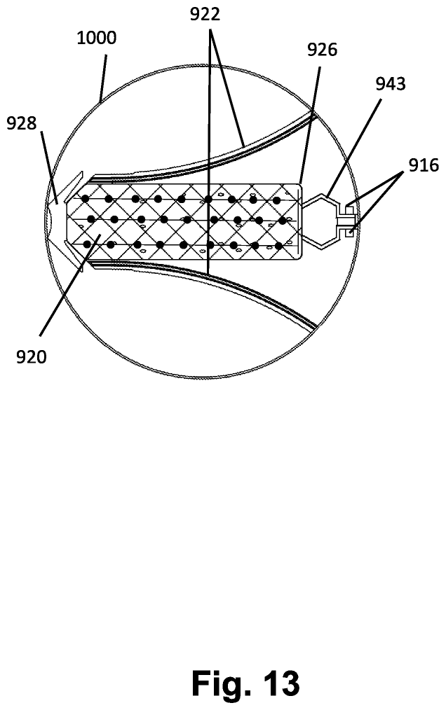

FIG. 12 and FIG. 13 illustrate one of these functional differences which is best described in the context of a method for transferring inoculation surface 920 to a petri dish. Some exemplary embodiments of the invention relate to a method for transferring an inoculation surface to a petri dish. In some exemplary embodiments of the invention, the method includes placing a distal end 928 of an inoculation surface 920 connected to a cap 910 against an inner wall of a petri dish 1000 and rotating cap 910 attached to inoculation surface 920 about a pivotal attachment axle (916, see FIG. 9; FIG. 10, FIG. 11) to disengage pivotal attachment socket (914, see FIG. 9; FIG. 10, FIG. 11) from the axle so that flexible wings 922 attached to inoculating element 920 assume an open configuration and secure inoculating element 920 in petri dish 1000. In the depicted embodiment, a flexible rhomboid element 943 forces axle 916 into contact with an inner wall of a petri dish 1000. In some embodiments rhomboid element 943 obviates a need for wings 922. Alternatively or additionally, in some embodiments rhomboid element 943 is positioned at distal end 928 of inoculation surface 920.

Although embodiments of the invention have been described by way of illustration, it will be understood that the invention may be carried out with many variations, modifications, and adaptations, without exceeding the scope of the claims.

1. A system for collection, sampling, culturing, and inspection of fluids, comprising a culture testing surface, suitable to be read in a reading apparatus, wherein said surface comprises stabilization element(s), suitable to maintain said surface in a desired position in a plate adapted to be processed by said reading apparatus.

2. The system according to claim 1, comprising:
a) a fluid collection container provided with a lid;
b) a sampling and culturing apparatus comprising a vessel, a cap, and an inoculating element;
wherein the culture testing surface is suitable to be inserted through an opening in the lid of said fluid collection container;
and wherein said culture testing surface being releasably attached to the cap of said sampling and culturing apparatus;
said culture testing surface being adapted to be inserted into the vessel of said sampling and culturing apparatus, and to be detached from the cap of said sampling and culturing apparatus.

3. The system according to claim 2, wherein the lid comprises an opening for evacuation test tube.

4-6. (canceled)

7. The system according to claim 2, wherein the cap of the sampling and culturing apparatus comprises releasing element(s), suitable to release the culture testing surface from said cap.

8. The system according to claim 2, wherein the cap further comprises gripping elements, suitable to grip the inoculating element.

9. The system according to claim 2, wherein the inoculating element comprises prongs.

10. The system according to claim 2, wherein the stabilization element(s) are wing-like strips that are attached to the culture testing surface.

11. The system according to claim 2, wherein the stabilization element(s) are a part of the culture testing surface.

12. (canceled)

13. The system according to claim 1, wherein the culture testing surface is divided into two or more sections.

14. The system according to claim 2, wherein the lid of the fluid collection container further comprises an opening adapted to receive a strip inserted therethrough.

15. The system according to claim 14, wherein the strip opening is provided with a one-way valve, suitable to be inserted with the strip through it and into the fluid collection container without removing the lid.

16. The system according to claim 2, wherein the vessel of the sampling and culturing apparatus comprises a projection at its top internal section, geometrically suitable to allow taking out the culture testing surface together with the inoculating element from the vessel, and forcing inoculating element to stay at the top section of the vessel when inserting the culture testing surface back into the vessel.

17. A method for collection, sampling, culturing, and inspection of fluids, comprising:
a) providing a culture testing surface, suitable to be read in a reading apparatus, wherein said surface comprises stabilization element(s), suitable to maintain said surface in a desired position in a plate adapted to be processed by said reading apparatus;
b) providing a fluid collection container provided with a lid, wherein the culture testing surface is suitable to be inserted through an opening in the lid of said fluid collection container;
c) providing a sampling and culturing apparatus, comprising a vessel, a cap, and an inoculating element, wherein said culture testing surface is releasably attached to the cap of said sampling and culturing apparatus, and wherein said culture testing surface is adapted to be inserted into the vessel of said sampling and culturing apparatus, and to be detached from the cap of said sampling and culturing apparatus;
d) positioning said inoculating element at the bottom section of said culture testing surface;
e) holding the cap of said sampling and culturing apparatus while it is attached to said culture testing surface along with said inoculating element, and pulling them out of the vessel of said sampling and culturing apparatus;
f) dipping the prongs of said inoculating element in a fluid sample; and
g) returning the assembly of said inoculating element, said culture testing surface, and the cap of said sampling and culturing apparatus back into the vessel of said sampling and culturing apparatus.

18. The method according to claim 17, further comprising performing an incubation process on the sampling and culturing apparatus containing the culture testing surface.

19. The method according to claim 17, further comprising detaching the culture testing surface from the cap of the sampling and culturing apparatus and from the inoculating element, and placing it on a surface.

20-21. (canceled)

22. An apparatus comprising:
(a) a cap with a reversible pivotal attachment socket and two engagement tabs on an inner surface thereof;
(b) an inoculating surface containing culture media and having a reversible pivotal attachment axle at a proximal end and two normally open flexible wings; and
(c) an inoculating element adapted to slideably travel from said distal end to said proximal end causing said wings to close and engage said engagement tabs.

23. The apparatus according to claim 22, comprising a flexible rhomboid member at one or both ends of said inoculation surface.

24. A method comprising:
(a) placing a distal end of an inoculation surface connected to a cap against an inner wall of a petri dish;
(b) rotating said cap attached to said inoculation surface about a pivotal attachment axle to disengage a pivotal attachment socket from said axle so that flexible members attached to said inoculating surface secure said inoculating element in said petri dish.

25. The method according to claim 24, wherein said flexible members are configured as wings which assume an open configuration upon disengagement of said axle from said pivotal attachment socket.

26. The method according to claim 24, wherein said flexible members are configured as rhomboid elements at one or both ends of said inoculation surface.
